(19) United States
(12) Patent Application Publication
(10) Pub. No.: US 2015/0105651 A1
(43) Pub. Date: Apr. 16, 2015

(54) SYSTEMS AND METHODS FOR MRI-BASED HEALTH MANAGEMENT

(71) Applicant: ZBH Enterprises, LLC, Plymouth, MN (US)

(72) Inventor: Chang-Jiang Zheng, Shoreview, MN (US)

(21) Appl. No.: 14/516,058

(22) Filed: Oct. 16, 2014

Related U.S. Application Data
(60) Provisional application No. 61/891,440, filed on Oct. 16, 2013.

(51) Int. Cl. A61B 5/00; A61B 6/03; A61B 6/00; G01R 33/48

(52) U.S. Cl. CPC A61B 5/486; G01R 33/4806; A61B 6/037; G01R 33/481; A61B 6/4417; G01R 33/4812; A61B 5/7275; A61B 5/0077; A61B 5/0035; A61B 5/7475; A61B 5/0013; A61B 5/0022; A61B 5/4866; A61B 5/7282; A61B 5/7465; A61B 2576/02; A61B 5/055
USPC 600/411; 600/407; 600/410; 600/425; 600/427; 600/476

(57) ABSTRACT

A method for improving health may include the steps of receiving patient input from a first patient regarding at least one parameter, scanning the first patient for a biomarker using an internal imaging machine, receiving a health plan for the first patient, accessing a database and searching the database for other patients having patient input similar to the first patient, predicting changes in the biomarker based on a patient having input similar to the first patient, and presenting the first patient with the predicted results. Follow-up monitoring may include the steps of receiving internal imaging data as a baseline for a patient undergoing a health plan to improve one or more biomarkers, measuring the patient for one or more biomarkers, estimating the patient's progress by comparing the measurement taken with the baseline internal imaging data, and presenting the patient with the estimated progress.

### CROSS-REFERENCE TO RELATED APPLICATIONS

This application claims priority to U.S. Provisional Patent Application No. 61/891,440, filed Oct. 16, 2013, which is hereby incorporated in its entirety

### FIELD OF THE INVENTION

The present disclosure relates to systems and methods for health management. Particularly, the present disclosure relates to assisting and motivating patients to select and/or adhere to a health plan. More particularly, the present disclosure relates to examining and estimating internal changes and potential internal changes using internal imaging data so that a patient may see the internal effects that a health plan may have on his or her health.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

The prevalence of chronic diseases, such as obesity, diabetes, high blood pressure, heart attack, stroke, atherosclerosis, fatty liver, and others has risen dramatically in society today. For most of these chronic conditions, no curative treatments are available. Proactive optimization of dietary and exercise regimen is often the most cost effective solution. Its overall effectiveness is clinically proven, and its practical implementation is associated with low risk for side effects. However, changing one's established habit and lifestyle is difficult. A high level of continuing motivation is often the key. Often times, such motivation is based on external appearance. For example, U.S. Pat. No. 7,328,119 discusses “diet and exercise planning and motivation including apparel purchases based on future appearance,” and thus focuses on the desired external appearance to motivate a person to adhere to a diet and exercise routine. Such programs focusing on an individual's external appearance, may disregard or simplify the importance of internal changes.

Internal imaging technology such as magnetic resonance imaging (MRI) or computed tomography (CT) scanning can measure and evaluate various portions of a person's internal body regions. CT or MRI may be able to see inside the human body and assess the condition of internal organs and tissues. This advantage can be demonstrated through several examples. For cardiovascular concerns, MRI technology can detect the presence of plaque in arterial blood vessels and in the correct circumstances see the condition of the heart itself. Similarly in terms of body composition, CT scans or MRI technology may allow for relatively accurate quantification of subcutaneous fat, visceral fat, and pericardial fat. Further, MRI technology can be used to detect a wide range of soft tissue and musculoskeletal conditions such as disc degeneration, ligament tears, severe cartilage loss in joints, stress fractures, and others.

Thus, there is a need in the art for processes and methods for using internal imaging to motivate individuals to choose and adhere to health plans, such as diet and exercise plans.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

In one or more embodiments, a method for improving health may include receiving patient input from a first patient regarding at least one parameter, scanning the first patient for a biomarker using an internal imaging machine, and receiving a health plan for the first patient. The method may also include accessing a database and searching the database for other patients having patient input similar to the first patient. The method may also include predicting changes in the biomarker based on a patient having input similar to the first patient and presenting the first patient with the predicted changes.

In one or more embodiments, a method for improving health may include receiving internal imaging data as a baseline for a patient undergoing a health plan to improve one or more biomarkers. The method may also include measuring the patient for one or more biomarkers and estimating the patient's progress by comparing the measurement taken with the baseline internal imaging data. The method may also include presenting the patient with the estimated progress.

In one or more other embodiments, a system for improving health may include an internal imaging machine for scanning a first patient for a biomarker, a database of patient information, and a computing device operably connected to the internal imaging machine and database. The computing device may include a receiving module configured to receive input regarding parameters of the first patient and a an internal image reading module configured to receive and interpret internal imaging data from the internal imaging machine. The system may also include a searching module configured to search the database for patients having parameters similar to the first patient's parameters and a modeling module configured to model the first patient's predictive biomarker changes. The system may also include a user interface whereby a user may access the system.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 is a schematic diagram showing an MRI machine and a database in communication with a computing device, according to one or more embodiments of the present disclosure.

FIG. 2 depicts a method for health management, according to one or more embodiments of the present disclosure.

FIG. 3 depicts a method for health monitoring, according to one or more embodiments of the present disclosure.

FIG. 4 depicts another method for health monitoring, according to one or more embodiments of the present disclosure.

### DETAILED DESCRIPTION

The present disclosure, in some embodiments, relates to systems and methods for assisting and motivating persons with respect to various dietary, exercise, or other regimens. In some embodiments, the systems and methods described may provide an interactive, iterative, “what-if” type of procedure, allowing a patient to see the current health condition of their various internal organs and bodily features, along with the value of certain biomarkers, and a realistic projection of what they may be able to achieve in terms of improvements to the condition of these organs, features, and biomarkers as a result of a planned course of action such as diet and/or exercise. This may allow patients to customize a diet, exercise, or other regimen that is both feasible to undertake and likely to produce satisfactory results for their individual needs. Continued tracking over time may allow patients to track their adherence to the regimen and measure progress toward their goal. The methods and systems described may also allow a patient to tailor a diet, exercise, or other regime to his or her unique physiological characteristics and limitations.

The systems of the present disclosure may be used by patients or medical professionals, including doctors, nurses, nurse practitioners, or others. A system of the present disclosure may provide for inputting a series of patient parameters and a health plan. The system may receive, and may also interpret in some embodiments, a patient's internal imaging scan, such as an MRI scan. The system may then access and search a database or databases for comparable patient data. According to some embodiments, the system may use the comparable patient data to predict and model a patient's possible or predicted results based on the comparable patient results.

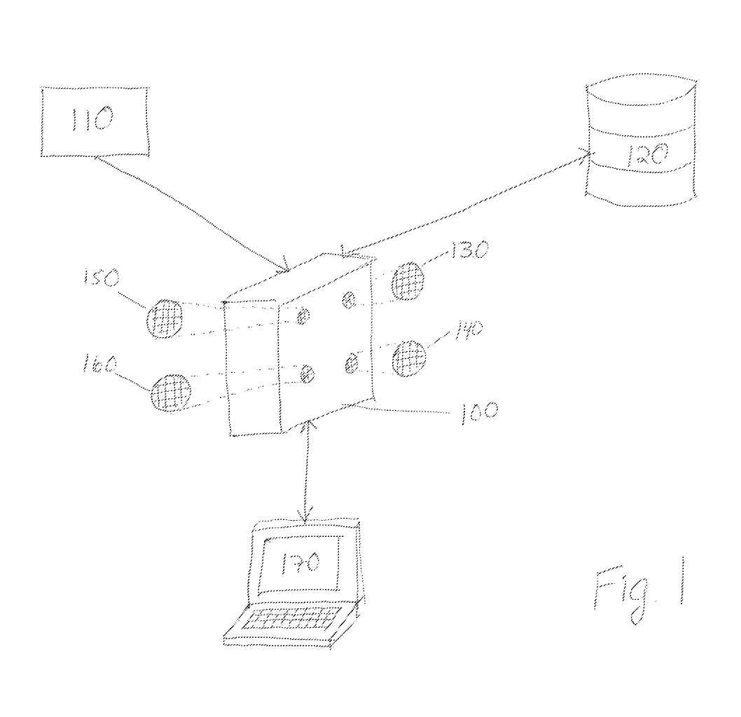

Referring now to FIG. 1, a computing device 100, available over a network to a user or at the same location of the user, is shown in communication with an MRI machine 110. The MRI machine 110 may be connected to the computing device 100 via a wired or wireless connection or network. In some embodiments, a CT or other internal imaging device may be connected to the computing device 100 instead of or in addition to an MRI machine 110. While an MRI machine 110 and MRI imaging data will be discussed herein, it is to be understood that any other internal imaging device and data or a combination may be used for the systems and methods disclosed herein. The computing device 100 may also be in communication via a wired or wireless connection with a database 120 or databases or a database may be included on the computing device 100.

The MRI machine 110 may be a standard or other machine used for conducting MRI scans. The machine 110 may be in the same location as the computing device 100 or may be at a remote location. The machine 110 may use low field or high field magnets to produce one or more images.

The database 120 or databases may contain patient data for any number of past or present patients. The database 120 may be located on the computing device 100 on an internal or external hard drive or memory device, or on the cloud or another network such as the Internet. The database 120 may include one or more parameters for each patient within the database. Patient parameters may include such information as age, weight, height, waist circumference, pain levels, medical history, or particular laboratory test results such as, for example, blood, proteomics, metabolomics/metabolites, genomics, or other test results. Still other laboratory test results and/or other patient parameters may be used. Patient parameters may also include current diet, current exercise, future diet, future exercise, desired health goals, or desired appearance goals. The database 120 may also include one or more health plans that each patient may have undergone. In some embodiments, the health plan may be a diet and/or exercise plan. The health plan may be a supplement routine, or other type of health plan. The database 120 may also include one or more MRI scans, data derived from MRI scans, or other MRI data for each patient. The scans or other imaging data may relate to other imaging devices such as CT scans in some embodiments. The database 120 may include a “before” MRI scan or data, performed before the patient began a health plan, along with an “after” MRI scan or data, performed after or near completion of the health plan or alternatively taken after adhering to the health plan for some time. MRI scans or data performed while the patient was at various stages of the health plan may be included in the database 120 as well. In some embodiments, the database 120 may include photographs for some patients, including for example, a “before” photo taken before the individual began the health plan, and an “after” photo taken after or near completion of the health plan or alternatively taken after adhering to the health plan for some period of time. The database 120 may include biomarker change data, showing changes each patient may have experienced by undergoing a health plan. Biomarkers may be, for example, visceral fat, muscle mass, arterial plaque, brain ventricle size, spine disc health, joint cartilage, or others. For example, if a patient saw a reduction in visceral fat after adhering to a certain diet plan for a period of time, the patient's visceral fat percentages calculated before beginning the diet plan and after adhering to the diet plan for a period of time may be included in the database 120. As new patient data is measured, scanned, or received, the new data may be input and stored in the database 120 so as to add to the quantity of patient data available in the database. As such, the database 120 may continue to grow in both size and sophistication because, in addition to new incoming patient data, new result-based data may be added allowing for further refining of health plans and/or treatment methods.

The computing device 100 may have certain modules, such as a data receiving module 130, an MRI reading or interpreting module 140, a searching module 150, and a modeling module 160, each configured to perform various processes. The computing device 100 may also have a user interface 170, whereby a user, such as a health professional, staff, or patient, can access and interface with the system. The computing device 100 and the several modules may include software, hardware, or a combination of software and hardware configured to perform a particular function. Moreover, while each element or module is described herein separately, each of them may be combined with one or more of the other elements or modules. The computing device 100 may also include a processor and computer readable storage medium.

The data receiving module 130 may be configured to receive information entered by a user. Such information may include one or more patient parameters and/or at least one health plan. In some embodiments, the data receiving module 120 may prompt a user for these inputs. Patient parameters may include such information as age, weight, height, waist circumference, pain levels, medical history, or certain laboratory test results such as, for example, blood test results. Patient parameters may be obtained from the patient or may be, in some cases, measured or tested or obtained from the patient's medical files, for example. In some embodiments, the health plan may be a diet and/or exercise plan. The health plan may be a supplement routine, or other type of health plan. The plan may be chosen with a goal of altering or improving one or more biomarkers. Biomarkers may be, for example, visceral fat, muscle mass, arterial plaque, brain ventricle size, spine disc health, joint cartilage, or others. The health plan may be chosen by the patient or by a medical professional in different embodiments.

The MRI reading module 140 may be configured to accept MRI data or images sent from the MRI machine 110. The MRI machine 110 may transmit results or readings from an MRI scan to the computing device 100, wherein the MRI reading module 140 receives the data. In some embodiments, the MRI reading module 140 may interpret the MRI readings. For example, the MRI reading module 140 may analyze a scan image to determine a percentage of visceral fat that a patient has or a percentage of cartilage loss or some other biomarker data. In some embodiments, multiple cross-sectional MRI images may be used to calculate a volume and/or weight of visceral fat, for example. In some embodiments, the MRI reading module may convert MRI data or result into a presentable or otherwise readable format. In some embodiments, the MRI reading module 140 may be capable of analyzing, comparing, or processing multiple MRI machine readouts or results simultaneously. The MRI reading module 140 may compare MRI data and present the comparison in readable form.

The searching module 150 may be configured to search the database 120 or databases using the information entered by a user and received via the data receiving module 130. The searching module 150 may search the database 120 for patients with one or more patient parameters in common with the information entered by the user. The searching module 150 may also or alternatively search by health plan or biomarker data. The searching module 150 may be configured to perform a search automatically after the data receiving module 130 receives information. In other embodiments, the searching module 150 may require confirmation or additional actions by the user before a search is performed. The searching module 150 may, according to some embodiments, permit a user to search through the database manually by viewing one or several entries at a time. The searching module may, thus, be capable of finding other patients with like characteristics or parameters thereby helping to predict the current patient outcome based on these similarities.

The modeling module 160 may be configured to prepare a model based on a comparison of input data received by the receiving module 130 and MRI data received by the MRI reading module 140, with patient data contained within the database 120 and found after the searching module 150 completes a search. That is, the modeling module 160 may compare a patient's one or more parameters, MRI data, photographs, biomarkers, and/or health plan with search results obtained by the searching module 150 in order to form a prediction as to any changes the patient may see by adhering to the health plan. For example, for a patient who wishes to lose visceral fat, the modeling module 160 may, by examining database records for other, similar patients who underwent a particular diet plan, estimate a percentage of fat loss that the patient may see after following the diet plan for a period of time. In some embodiments, the modeling module 160 may alter or update a prediction. Over time, as new or updated patient information is input and stored in the database 120, the searching module 150 may perform a new search, providing updated patient information from which the modeling module 160 can prepare a model. That is, as new searches are performed, the modeling module 160 may alter the projected model to reflect any changes in the data returned by the searching module 150. It is to be appreciated that the database 120 may be a growing database with respect to the number of patients being treated and, thus, the amount of data being stored. However, in addition, the database 120 may also increase in sophistication as new results reflect more efficient and/or effective treatment plans for particular patient types. The modeling module 160 may present the predicted result in terms of numbers or charts such as, for example, a number or graph showing a change in visceral fat. In some embodiments, the modeling module 160 may show the predicted result as an MRI scan, mock MRI scan, or MRI scan with drawn in changes. The modeling module 160 may similarly show the predicted result as a photograph, mock photograph, or photograph with drawn in changes. In further embodiments, the modeling module 160 may present the predicted changes as a two-dimensional or three-dimensional model. In any of these two-dimensional or three-dimensional graphical depictions, the modeling module may reduce the amount of visceral fat in an organ, for example, by reducing the area or volume shown in an image. The modeling may be automatically performed after a search of the database 120 results in one or more predictive outcomes, or may require user inputs or actions. In some embodiments, multiple photographs may be taken of the patient at a variety of angles in order to better render a two-dimensional or three-dimensional model of the patient's body. The external photographs may be combined with the internal MRI data to create a more accurate model, reflecting internal and external conditions. The modeling module 160 may then project predicted changes onto that model in some embodiments.

In operation, the system may perform various methods. One such method 200 may be used during an initial patient visit, for example, to help establish a health plan. The method may generally include capturing data about the patient, defining a biomarker or markers on which motivation and progress tracking will be based, establishing a health plan for targeting such biomarker or other health improvement, and predicting an overall outcome. The details of the method are discussed in more detail below.

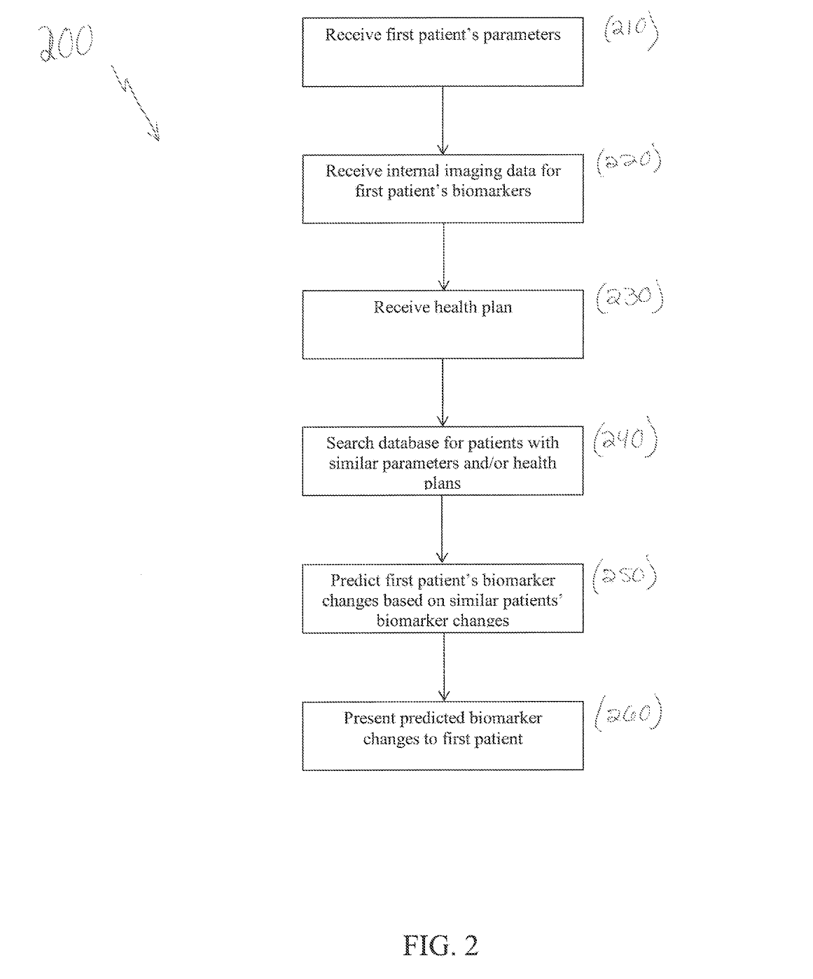

Referring now to FIG. 2, the method may include obtaining and/or receiving one or more patient parameters. (210) As described above, patient parameters may include such information as age, weight, height, waist circumference, pain areas and levels, medical history, or certain laboratory test results such as, for example, blood test results. Patient parameters may be obtained from the patient or may be, in some cases, measured or tested or obtained from the patient's medical files, for example. The above information may be collected for at least two reasons. First, this information may provide a physician or other health plan agent with general information about a patient's health. As such, this information may help identify relevant biomarkers that may be useful to help monitor and/or track progress. Second, this information may be useful for purposes of predicting outcomes as discussed in more detail below. The computing system 100 of FIG. 1 may receive this information via manual entry or via download from other electronic sources such as electronic medical records, or other databases, for example.

In addition to patient parameters, the method may also include receiving and/or obtaining MRI or other internal imaging scan or imaging-based data. (220) The imaging data may be targeted imaging data that is focused on a previously defined biomarker. For example, where the patient parameters suggest that a patient is at risk of heart disease or other cardiovascular disorder, imaging data may be obtained of abdominal visceral fat, arterial plaque, or another biomarker indicative of these conditions. As another example, where patient parameters suggest low back pain, knee pain, or some other type of pain, imaging data of biomarkers that are indicators of these items may be obtained. For example, imaging data of spinal discs or knee cartilage may be obtained and/or provided. Obtaining this imaging data of one or more biomarkers may establish a baseline for further monitoring and tracking of progress relating to the health plan. This imaging data may also be helpful in modifying, supplementing, or otherwise modifying the health plan to expedite results or otherwise efficiently address patient issues or concerns. For example, where severe conditions are revealed, surgery or other procedures may be recommended in lieu of or in addition to a related health plan. The computing system 100 of FIG. 1 may receive or obtain the above biomarker information via the receiving module 130 by interacting with an MRI machine 110, other imaging device, or a database 120, for example. In some embodiments, particular MRI scanning protocols may be used. One example of such protocols has been presented below.

A patient health plan may also be established and the plan may be obtained and/or received by the computing system 100 via manual entry or via details of a plan that may be stored in a database, for example. (230) In some embodiments, the health plan may be a diet and/or exercise plan. The health plan may be a supplement routine, or other type of health plan. The plan may be chosen with a goal of altering or improving one or more of the above-mentioned biomarkers. As described above, biomarkers may be, for example, visceral fat, muscle mass, arterial plaque, brain ventricle size, spine disc health, joint cartilage, or others. In some embodiments, a medical professional may select a health plan for the patient. In some embodiments, the selected health plan and the biomarkers may be selected such that continued monitoring and/or tracking of the biomarker may be reflective of how well the patient is adhering to the health plan. As such, in some embodiments, a health plan and biomarker combination may include a diet that is particularly low in carbohydrates together with monitoring and tracking of abdominal visceral fat. In other embodiments, a health plan and biomarker combination may include an exercise plan adapted for weight loss and stomach muscle strengthening together with a monitoring and tracking of spinal disc condition.

With a goal of predicting the outcome of the health plan, the system 100 may search for like-situated patients using the search module 150. For example, the system may search for other patients with the same or similar patient parameters, baseline biomarker results, and/or health plans. (240) Depending on the nature of the patient and the experience of the provider, particular parameters may be given higher priorities where the provider believes or understand such parameters to have a relatively high correlation to biomarker changes. For example, a patient that is male, weighs, 275 pounds, is 6′-0″ tall, and has 10.0 pounds of abdominal visceral fat, may have a relatively high propensity for heart disease or other cardiac disorder. When the system searches for like-situated patients, the system may be set to give a higher priority to the abdominal visceral fat value when searching for like-situated patients. In other cases, a particular number of parameters may be selected for searching for like-situated patients. For example, the user may set the value at 3 parameters, 10 parameters, or other parameter values. Accordingly, where the database includes a high number of like-situated persons, the user may adjust the results and hone in, so to speak, on a subset of persons that are quite like the patient being treated. On the other hand, when the database includes a low number of like-situated persons, the number of parameters used to find like-situated persons may be lowered to obtain a sufficient number of persons for comparison. The user may interact with the system 100 to review the results of the search and adjust the search criteria to obtain a subset of comparable patients.

Based on the search results, the patient's biomarker changes for adhering to the health plan may be predicted. (250) For example, if the search of step 240 resulted in 10 like-situated patients, and if the biomarker is abdominal visceral fat, the patient's visceral fat loss over a particular period of time may be predicted by averaging or otherwise collating or combining the visceral fat loss of the like-situated patients over the same time period and potentially following the same or similar health plan. In the case of back pain patients, for example, a predicted slowing and/or halting of disc degeneration may be predicted. Still other biomarker change predictions may be made by reviewing results from like-situated patients. The system 100 may receive input relating to the desired biomarker or biomarkers as well as other information such as the period of time over which the prediction is based and any other information used to establish the prediction. The system may capture the result for each of the subset of comparable patients and may attend to averaging, collating, or otherwise combining such biomarkers to establish a prediction. The system 100 may predict short-term and/or long-term results. For example, in one embodiment, the system 100 may, by reviewing results from like-situated patients, forecast a six-week benefit that a patient may see as well as a five-year overall health benefit that a patient may see if the patient adheres to the particular health plan.

The predicted biomarker changes may also be presented (260). In some embodiments, such changes may be presented in numeric form such as an expected amount of visceral fat to be lost. In other embodiments, a graph of expected visceral fat loss over time may be presented. In still other embodiments, a two-dimensional or three-dimensional model may be used to depict the physical changes. For example, the modeling module 160 may adjust a patient's image data by reducing the amount of visceral fat shown in an image. For example, where a patient has 10 pounds of visceral fat and it is predicted that they will lose 3 pounds of visceral fat over a particular time period, the image data may be adjusted to reflect a 30% decrease in visceral fat. This modeling may be performed for selected cross-sections of MRI data, for example, or the modeling may be performed for all of the cross-sections. In still other embodiments, three-dimensional modeling may be used to develop before and after images based on the predicted changes. In some embodiments, a health professional may draw on or mark up, either manually or digitally, the patient's internal imaging scan and/or external photographs to present the predicted changes to the patient. In some embodiments, the system may allow the user to toggle between different health plans or edit and adjust particular aspects of the health plan to quickly adjust the predicted biomarker changes. As these health plans or particular aspects are adjusted, the system may adjust the search and prediction, as needed, to obtain suitable results and adjust the presentation accordingly. As such, a user may be able to refine a health plan based on patient goals and desires and may be able to motivate patient to select a more aggressive health plan based on improved results.

The method 200 described may provide the patient with an opportunity to see parameters, internal imaging data, biomarker data, and/or photographs that illustrate the initial, interim, and/or final stages of health plans. By comparing similar health plans and similar parameters, a patient may be able to see a predictive outcome of the success a health plan may have for his or her particular needs, based on the outcome that such a health plan had on other similarly situated individuals. The method may provide not only an incentive to select a health plan in order to move toward a goal, but also motivation to remain on the health plan due to visualization of actual results in similarly situated individuals. By comparing a patient's internal imaging data to imaging data of similarly situated individuals, a patient may be able to see the internal benefits that a diet, exercise, supplement, or other regime may have on his or her body. In some embodiments, the focus of the method may be on internal benefits and internal imaging, such that a patient may be motivated by the possible internal benefits to his or her tissues or organs, and in such cases, photographs illustrating external changes may not be used or compared. In some embodiments, the health plan may be one of the inputs used to search the database, as described above. In other embodiments, a patient or health professional may search the database without inputting a health plan, and then select an appropriate health plan based on the results of the search. Still other orders of steps in the method may be adjusted or changed.

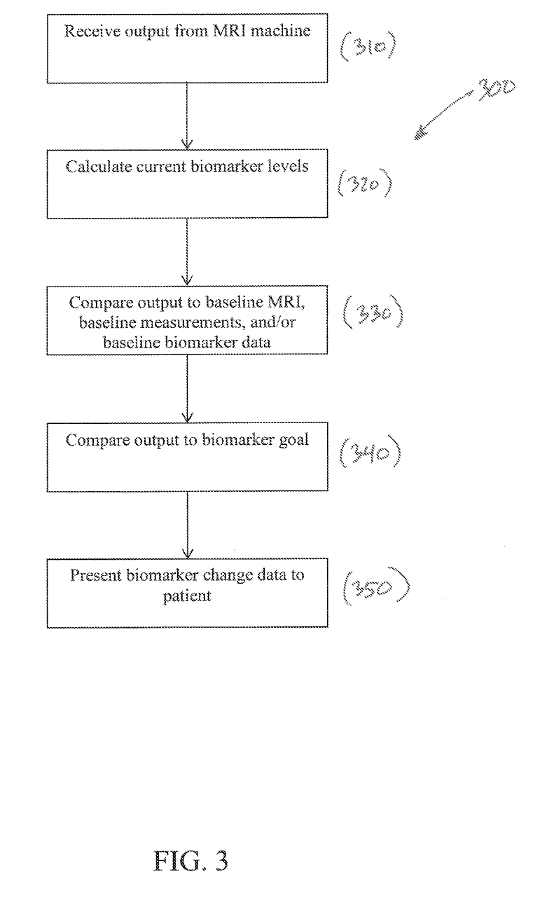

In some embodiments, a patient's progress may be continuously monitored while the patient undergoes the elected health plan(s). For example, after a patient undergoes the initial input and search process and elects a health plan based on predicted biomarker changes, the patient may return for follow-up measurements to track his or her progress toward a biomarker goal. FIG. 3 illustrates a method 300 of tracking such progress.

A patient's progress while executing a health plan may be tracked by using the MRI machine 110, or other internal imaging. As shown in FIG. 3, a patient may return for a follow up visit and a follow-up MRI may be performed or output from an MRI machine 110 may otherwise be obtained. (310) That is, an MRI may be performed at the facility or a third party MRI may be performed and the data stored and/or sent to system 100 for analysis. The MRI data may be targeted to examine or calculate a biomarker, such as the biomarker or biomarkers that the patient is attempting to change by undergoing the health plan. For example, if the biomarker is visceral fat, an MRI of the carotid artery or the abdominal area, for example, may be performed to capture image data of visceral fat. The resulting image data may be stored in a database 120.

The system 100 may analyze the image data to calculate current biomarker values. (320). That is, for example and as with the baseline calculation, multiple cross-sections of a portion of the body may be captured and the areas of visceral fat, for example, may be used to calculate a volume or weight of visceral fat. Similar approaches may be used to perform calculations and establish quantified values reflecting changes in visceral fat, disc condition, cartilage condition, and other biomarker changes.

The resulting values may be compared to the baseline (330). This may allow a user and/or patient to have an idea of the progress that is being made as a result of the executing the health plan. In some embodiments, the system 100 may perform a calculation to establish the difference between the baseline value and the current value of the biomarker. For example, where the initial value of visceral fat was 10 pounds and the current value is 7 pounds, the resulting change may be the difference of 3 pounds of visceral fat that has been lost.

In addition to comparing to the baseline values, the current biomarker values may be compared to the biomarker goal or target that the patient hopes to achieve, so as to track the patient's progress toward the goal. (340) That is, if the goal is to lose 8 pounds of visceral fat in 16 weeks, and the patient returns for a 4 week checkup having lost 2 pounds of visceral fat, the patient may be said to be on track. In other cases, larger quantities may be anticipated to be lost in the early stages of a health plan and, as such, the above patient may be said to be behind schedule.

For any and/or all of the above comparisons, numerical, graphical, or image-based information may be provided to the patient to explain this progress. (350). For example, with respect to visceral fat, a total amount of visceral fat lost or a percentage lost may be presented. In addition, a graph showing progress of visceral fat lost over time and in comparison to the health plan period may be provided. Still further, MRI image data or outlines thereof may be overlaid to show patients visually how much visceral fat has been lost. Still other approaches to showing the patient their progress and explaining the effect on their condition may be used. As suggested above, method 300 may be performed in conjunction with method 200 where method 200 may be used to establish a baseline, implement a health plan, and show predicted outcomes and method 300 may be used to update a patient on their progress during execution of a health plan.

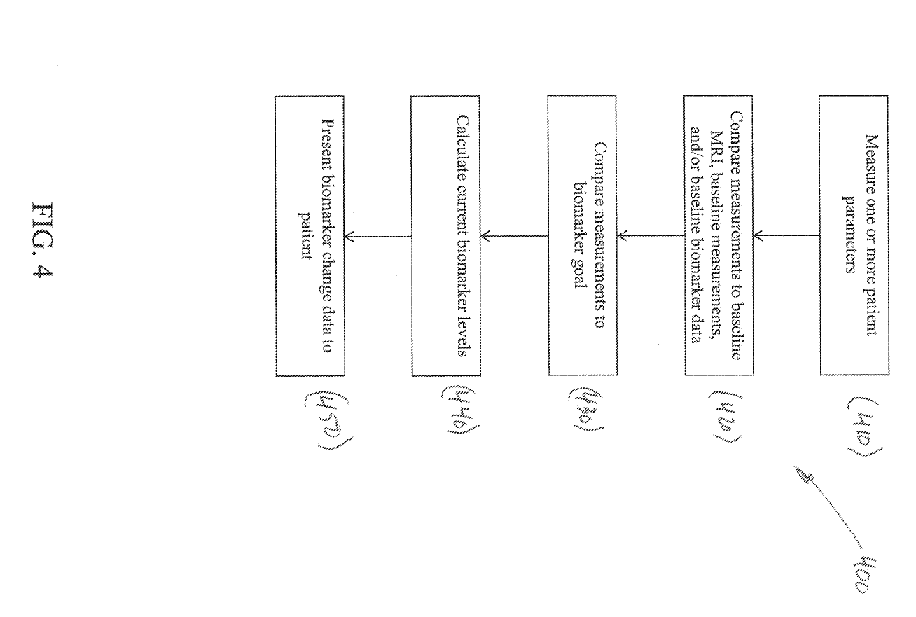

FIG. 4 shows another method of tracking a user's progress during execution of a health plan. The method 400 is based on the idea that changes in patient parameters may be correlative to patient biomarkers and measurement of patient parameters may be suitable for assessing the progress of a patient executing a health plan.

As shown in FIG. 4, one or more patient measurements may be taken. (410) Such measurements may include parameters such as weight, waist circumference, or pain level, for example. Measurements may also include blood test data or other measured data. Still other measurements may be included and, in particular, may include measurements that are believed to be correlative to changes in biomarker values.

The measurements that are taken may be compared to previous respective measurements, MRI data, and/or biomarker data received before the patient began the health plan. (420) That is, the current data may be compared to previous data so as to calculate any changes in the data thus far. Such changes may be in the form of percentage changes or absolute values may be used. The form that the changes are presented in may be whichever form is relevant to a corresponding change in a biomarker value.

The measurements that are taken may also be compared to the target that the patient hopes to achieve, so as to track the patient's progress toward the goal. (430) That is, while the goals of the plan may be based on a biomarker, such goals may also be correlative to, for example, waist size. As such, while method 200 above discusses predicting biomarker values, patient parameters such as waist size or other parameters may also be predicted allowing progress toward the goal to be defined, in part, by changes in the respective parameter.

The compared differences between the initial measurements, target biomarker values, and current measured data may then be used to calculate current biomarker levels. (440) In some embodiments, the current biomarker levels may be estimated based on starting biomarker levels, MRI data, and parameters, as compared with current measurements. In other embodiments, the current measured parameters may provide enough information to estimate or calculate one or more current biomarker levels. For example, if a patient's overall weight was reduced by 5% between the baseline value and the current value, a corresponding reduction in a visceral fat biomarker may also be determined. Similarly, if the above 5% overall weight loss reflects 10% of the weight a patient expects to lose on the plan, the system may calculate the value of a 10% reduction in the amount of visceral fat that was to be lost on the plan and calculate a biomarker value in that manner. It is noted that either or both of the above correlations may be straight comparisons, where the percentage reduction in weight loss is the same percentage reduction in visceral fat or experience may suggest that the relationship between weight loss and visceral fat reduction is not linear. Rather, a reduction of 5% in overall weight may reflect another percentage value loss of visceral fat. These correlations may be developed over time and may allow a change in a patient parameter to help define a current biomarker value without the need to perform additional MRI scans, for example.

As with methods 200 and 300, the current biomarker values may be presented to the patient in an understandable form such as with numbers, graphs, images, etc. (450) Method 400 may be used in conjunction with method 200 or similar methods. Method 400 may also be used with method 300 such that the two methods can be used to cross-check on another and/or method 300 may be used to double check method 400 unless/until method 400 becomes more reliable. Thus, a patient may begin with a baseline MRI scan, and the patient's progress may be tracked by comparing bodily measurements to the baseline MRI and the target result. In this way, internal imaging data may be leveraged in a way that provides a cost-effective motivational tracking tool for patients.

In some embodiments, the methods 300, 400 may be performed together. That is, ongoing measurements and MRI scans may be used to track a patient's progress while undergoing a health plan. The patient's progress may be tracked, and thus one or both methods 300, 400 may be performed, on a daily, weekly, monthly, or other interval. In some embodiments, for example, follow-up measurements and MRI scans may be conducted every six weeks or three months. The interval need not be consistent. For example, a patient's progress may be tracked more frequently at the initial stages of a health plan and less frequently as the patient progresses. In some embodiments, the methods 300, 400 may be performed at different rates. For example, a patient's progress may be tracked by measurements each month, while the patient's progress may be tracked by new MRI scans every six months.

By seeing the results of follow-up measurements and/or follow-up MRI scanning, a patient may feel motivated to continue adhering to the one or more health plans. As a patient can continually see the effects that the health plan(s) are having on his or her body, particularly with respect to the changes in biomarker levels that the patient may be attempting to improve, the patient may be motivated to continue the progress. The methods 300, 400 may allow a patient to understand the effects of the health plan on an internal level. Specifically, by viewing changed biomarker data and/or changed MRI data, a patient may understand the differences occurring in his or her internal organs and/or tissues. The tracking methods 300, 400 may also allow a patient and/or health professional an opportunity to tailor the patient's one or more health plan when needed such as in response to the changes seen thus far or in order to achieve a new biomarker goal. In some embodiments, external photographs may also be used with the methods 300, 400. Photographs may be taken in addition to follow-up measurements or follow-up MRI scanning. The photographs may be compared to past photographs and/or predictive modeling data. The external changes viewed in the photographs may provide further motivation for patients.

In conjunction with the above methods 300, 400, the system 100 may perform one or more follow-up database 120 searches for like-situated patients at intervals while the patient's progress is tracked. The searching module 150 may perform a new search based at least in part on updated measurements, parameters, image data, and/or biomarker data obtained from the patient. Or in some embodiments, a new search may be performed based on previously obtained measurements, parameters, image data, and/or biomarker data, thus in some cases repeating a previously performed search. The results of a follow-up search may differ from any previously performed searches due to the current patient's newly measured or calculated data providing new search parameters. The results of a follow-up search may also differ from any previously performed searches due to other patients' updated data and results having been input and stored in the database 120 since the previous search. Follow-up search results may be used to suggest or establish a new health plan, for example if the patient's progress and follow-up search results suggest that the patient may be better suited to a different health plan, or may be used to suggest or establish a new or additional biomarker goal. As may be appreciated, the database 120 and associated system-suggested client counseling and instruction may continually evolve and/or learn over time based on the continued input of additional patient data and results together with treatment leading to those results. Accordingly, as a patient undergoes counseling along a health plan, the growing database 120 may create a higher number or a more comparable set of comparable patients on which the patient's plan may be based. In addition, the results of these or other patients in the database 120 may reflect a need or opportunity to modify, change, or otherwise alter a patient's health plan to more effectively or efficiently reach their goal. Still further, the updated search may reflect added or confirmed benefits (i.e., anticipated likelihood of overcoming diabetes, or anticipation of a pain free back, etc.) of the plan the patient is already on, providing for additional motivation or encouragement to stick to the plan. As such, the system 100 may reflect an artificial intelligence system that continually grows in size and sophistication and thus has an ability to continually adjust its suggested treatment plans.

In some embodiments, MRI scanning or other internal imaging may be conducted in a cost-effective manner. Typically, internal image scanning can be prohibitively expensive for some patients, particularly when multiple MRI scans are performed at intervals. In some embodiments, low field magnets may be used instead of high field magnets. Low field magnets may require longer scan times and produce images with lower clarity or resolution than high field magnets, but generally may provide sufficient imaging to examine and analyze certain biomarker data, such as visceral fat or spinal disc degeneration. Low field magnets may generally be less expensive than high field magnets. Low field magnets also may reduce shielding requirements which may further reduce costs. Further, simplified MRI protocols with fewer slices may be performed to counteract the long scan times associated with low field magnets. The following protocols provide some examples of simplified protocols that may be used to examine or analyze certain biomarkers. These examples are for illustrative purposes, and MRI protocols used to perform the methods herein need not be restricted to them.

Visceral Fat—Abdomen

Simplified, adaptive screening exam using a low-field MRI (0.3 T)

Simplified:
Two T1 axial sequences; each completed within one breath holding session (about 20 seconds)
First axial sequence consists of 5 axial slices; centered on L3-L4
Second axial sequence consists of 5 axial slices; centered on the mid-vertebral body of L3

Adaptive (with or without physician supervision):
If lesion in major organ is incidentally noticed, add fat-suppressed protocol

Detailed Specifications:
First Axial sequence:
FOV 420
TR 217
TE 25
FA 90
Slices 5
Thickness 10 mm
Gap 20 mm
Frequency 256
Phase 160
Half scan
Signal average 1
Breath hold 24 seconds
Second Axial sequence:
FOV 420
TR 217
TE 25
FA 90
Slices 5
Thickness 10 mm
Gap 20 mm
Frequency 256
Phase 160
Half scan
Signal average 1
Breath hold 24 seconds

Visceral Fat—Chest

Simplified, adaptive screening exam using a low-field MRI (0.3 T)

Simplified:
T1 axial
Very short TR
No breath holding is necessary
Scan begin from above the aorta

Adaptive
If lesion in major organ is incidentally noticed, add fat-suppressed protocol

Detailed specifications:
FOV 420
TR 610
TE 25
Flip angle 90
Slices 14
Thickness 10 mm
Slice gap 2 mm
Frequency 256
Phase 172
Half scan
Signal average 4

Low Back and Neck

Simplified, adaptive screening exam using low-field MRI scanner (0.3 T)

Simplified:
T2 sagittal view

Adaptive (with or without physician supervision):
If disc bulging is noticed, add T1 sagittal, T1 axial, T2 axial
If modic change is noticed, add T1 sagittal, T1 axial, T2 axial

Detailed Specifications
Axial/Sagittal/Coronal localizer
Sag T2
FOV 300
TR 4000
TE 100
FA 90
Slices 11
Thickness 5 mm
Gap 1.5 mm
Frequency 256
Phase 212
Signal average 6

Knee Screening Exam

Simplified, adaptive screening exam using a low-field MRI (0.3 T)

Simplified:
T1 coronal (cartilage thickness evaluation)
T1 axial (knee cap alignment)

Adaptive (with or without physician supervision):
If meniscal tear is noticed, add T2 sagittal and coronal STIR view
If bony lesion noticed, add STIR coronal view
If increased resolution is needed to evaluate tibial/femoral cartilage, add 3D coronal T1

Detailed Specifications:
Knee axial/sag/cor localizer
Coronal Proton Density:
   FOV 180
   TR 1000
   TE 25
   FA 90
   Slices 22
   Thickness 3.5 mm
   Gap 1.0 mm
   Frequency 256
   Phase 200
   Half scan
   Signal average 1
Axial Proton Density:
   FOV 180
   TR 1000
   TE 25
   FA 90
   Slices 22
   Thickness 3.5 mm
   Gap 1.0 mm
   Frequency 256
   Phase 200
   Half scan
   Signal average 1
3D Coronal T1
   FOV 170
   TR 46
   TE 18
   FA 90
   Enclosed Slab 36
   Multi slab number 1
   Angle number 1
   Thickness 3.0 mm
   Interval 0.0 mm
   Frequency 256
   Phase 200
   Half scan OFF
   Signal average 1

Carotid Artery Screening

Simplified, adaptive screening exam using low-field MRI (0.3 T)

Simplified:
2D axial time of flight MRA of carotid artery

Detailed Specifications:
2 plan localizer: coronal and axial
   FOV 260
   TR 150
   TE 25
   FA 90
   Multi slice number 6
   Multi echo number 1
   Angle number 2
   Thickness: 7.0 mm
   Interval: 2.0 mm
   Frequency 256
   Phase 128
   Half scan ON
   Signal average 1
2d Axial time of flight MRA of carotid artery
   FOV 180
   TR 45
   TE 9
   FA 90
   Multi slice number 50
   Multi echo n/a
   Angle number n/a
   Thickness 3.0 mm
   Interval 2.0 mm
   Frequency 256
   Phase 180
   Half Scan OFF
   Signal average 1
T1 Black blood protocol
   Orientation axial
   Spin echo
   FOV 180
   TR 1000
   TE 28
   FA 90
   Multi slice 10
   Thickness 3.0 mm
   Interval 3.0 mm (0 mm gap)
   Freq 256
   Phase 192
   Half scan: OFF
   Dual slice: ON
   Signal average 6

The use of an MRI machine 110 for continuous monitoring, as compared with other internal imaging methods such as X-rays or PET scans, may be a decreased exposure to radiation. An added benefit of continuous MRI scanning is to monitor for and possibly detect early any new health concerns or problems. In some embodiments, method 300 or similar iterative MRI scanning methods may be used for screening purposes to continually monitor for certain diseases, illnesses, conditions, or other health problems. For example, if a patient is at risk for a particular condition, the use of a baseline MRI scan combined with iterative scanning may allow continued tracking and monitoring of the concerns. In some cases, such iterative monitoring may be combined with one or more health plans so as to reduce the patient's risk of developing a condition, for example. The use of low field magnets and/or simplified protocols may allow for cost-effective monitoring in this manner. In further embodiments low cost MRI screening protocols may be used for such applications as pre-employment physicals, employee wellness, executive wellness, personalized wellness, personalized fitness training, personalized sports injury prevention, personalized adaptive fitness coaching, personalized adaptive wellness coaching, and others.

The systems and methods described herein may be beneficial in helping patients improve a number of biomarkers. Following are some illustrative examples of some of the biomarker data that may be examined and analyzed using the methods and systems described. These examples are for illustrative purposes, and the methods and systems disclosed herein may be used to examine, analyze, and assist patients with improving any number of bodily issues.

Visceral Fat with Baseline MRI and Follow-Up Measurements

The systems and methods may be used to assist and motivate a patient to reduce visceral fat content. In one example, a baseline MRI scan may be combined with biophysical measurements and laboratory-based biomarkers to track the internal change and predict reduction of visceral fat over time. Before or after a baseline MRI scan is performed, a patient may elect a health plan such as a diet plan. Volume and density calculations may be used to calculate or estimate the patient's starting visceral fat levels based on the baseline MRI scan. Thereafter, while the patient undergoes the diet plan, follow-up measurements may be taken at intervals. For example, the patient's change in weight, waist circumference, and/or triglyceride levels may be measured. Such measurements may be predictive of visceral fat content in some cases. These or other biophysical measurements may be used to estimate the patient's change in visceral fat content. Estimated changes in visceral fat content may be presented to the patient so as to motivate the patient to continue adhering to the diet plan.

Spinal Disc Degeneration with Baseline MRI and Follow-Up MRI's

The systems and methods may be used to assist and motivate a patient to slow or stop spinal disc degeneration, thereby reducing pain in some cases. In one example, a baseline MRI scan may be combined with an exercise plan and follow-up MRI scans taken at intervals to assess and monitor/motivate an individual to slow or otherwise correct disc regenerations. Other parameters such as weight loss or pain level, which may indicate progress in slowing or halting disc degeneration, may be monitored at intervals as well.

In still other examples, similar approaches may be used to address issues of joint cartilage, pericardial fat, and the like.

Systems and methods for assisting and motivating persons with respect to various dietary, exercise, or other regimens have been disclosed. In some embodiments, the systems and methods described may provide an interactive, iterative, “what-if” type of procedure allowing patients to view the state of their current health or certain biomarkers, and projections of what they could achieve by adhering to one or more health plans. In some embodiments, projections may be based on a database of other patient information. In some embodiments, the systems and methods described herein may be used to build or acquire a database of patient information that may be used to find suitable health plans for future patients. Continued monitoring over time may allow patients to track their adherence to the regimen and measure progress toward their goal. The methods and systems described may also allow a patient to tailor a diet, exercise, or other regime to his or her unique physiological characteristics and limitations.

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a personal computer (e.g., desktop or laptop), tablet computer, mobile device (e.g., personal digital assistant (PDA) or smart phone), server (e.g., blade server or rack server), a network storage device, or any other suitable device or combination of devices and may vary in size, shape, performance, functionality, and price. A system may include random access memory (RAM), one or more processing resources such as a central processing unit (CPU) or hardware or software control logic, ROM, and/or other types of nonvolatile memory. Additional components of a system may include one or more disk drives or one or more mass storage devices, one or more network ports for communicating with external devices as well as various input and output (I/O) devices, such as a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, or any combination of storage devices. A system may include what is referred to as a user interface, which may generally include a display, mouse or other cursor control device, keyboard, button, touchpad, touch screen, microphone, camera, video recorder, speaker, LED, light, joystick, switch, buzzer, bell, and/or other user input/output device for communicating with one or more users or for entering information into the system. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices. A system may also include one or more buses operable to transmit communications between the various hardware components.

One or more programs or applications, such as a web browser, and/or other applications may be stored in one or more of the system data storage devices. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information.

Hardware and software components of the present disclosure, as discussed herein, may be integral portions of a single computer or server or may be connected parts of a computer network. The hardware and software components may be located within a single location or, in other embodiments, portions of the hardware and software components may be divided among a plurality of locations and connected directly or through a global computer information network, such as the Internet.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language, Fortran language, or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart may illustrate a method as a sequential process, many of the operations in the flowcharts illustrated herein can be performed in parallel or concurrently. In addition, the order of the method steps illustrated in a flowchart may be rearranged for some embodiments. Similarly, a method illustrated in a flow chart could have additional steps not included therein or fewer steps than those shown. A method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A method for improving health, comprising:
receiving patient input from a first patient regarding at least one parameter;
scanning the first patient for a biomarker using an internal imaging machine;
receiving a health plan for the first patient;
accessing a database and searching the database for other patients having patient input similar to the first patient;
predicting changes in the biomarker based on a patient having input similar to the first patient; and
presenting the first patient with the predicted changes.

2. The method of claim 1, wherein scanning the first patient for a biomarker comprises scanning the patient for a plurality of biomarkers.

3. The method of claim 1, wherein scanning the first patient for a biomarker comprises scanning the health of an organ.

4. The method of claim 1, wherein the one or more parameters include one or a combination of race, gender, age, current diet, current exercise, future diet, future exercise, desired health goals, desired appearance goals, and physical information.

5. The method of claim 1, wherein scanning of the first patient is performed with at least one of low-field MRI, mid-field MRI, high-field MRI, functional MRI, CAT, PET, and xRAY.

6. The method of claim 1, further comprising capturing one or more external camera images of the first patient.

7. The method of claim 1, further comprising selecting the health plan based on health plans chosen by patients having input similar to the first patient.

8. The method of claim 7, wherein the health plan is iterated and modified based on results.

9. A method for improving health, comprising:
receiving internal imaging data as a baseline for a patient undergoing a health plan to improve one or more biomarkers;
measuring the patient for one or more biomarkers;
estimating the patient's progress by comparing the measurement taken with the baseline internal imaging data; and
presenting the patient with the estimated progress.

10. The method of claim 9, wherein the internal imaging data comprises a scan from at least one of low-field MRI, mid-field MRI, high-field MRI, functional MRI, CAT, PET, and xRAY.

11. The method of claim 9, wherein measuring the patient for one or more biomarkers comprises taking one or more biophysical measurements.

12. The method of claim 9, wherein measuring the patient for one or more biomarkers comprises scanning the patient using at least one of low-field MRI, mid-field MRI, high-field MRI, functional MRI, CAT, PET, and xRAY.

13. The method of claim 1, wherein the health plan is iterated and modified based on results.

14. A system for improving health, the system comprising:
an internal imaging machine for scanning a first patient for a biomarker;
a database of patient information;
a computing device operably connected to the internal imaging machine and database, the computing device comprising:
a receiving module configured to receive input regarding parameters of the first patient;
an internal image reading module configured to receive and interpret internal imaging data from the internal imaging machine;
a searching module configured to search the database for patients having parameters similar to the first patient's parameters;
a modeling module configured to model the first patient's predictive biomarker changes; and
a user interface whereby a user may access the system.

15. The system of claim 14, wherein the parameters include one or a combination of race, gender, age, current diet, current exercise, future diet, future exercise, desired health goals, desired appearance goals, and physical information.

16. The system of claim 14, wherein the internal imaging machine comprises low-field MRI, mid-field MRI, high-field MRI, functional MRI, CAT, PET, or xRAY.

17. The system of claim 14, further comprising selecting a health plan based on health plans chosen by patients having input similar to the first patient.

18. The system of claim 17, wherein the health plan is iterated and modified based on results.

19. The system of claim 14, wherein the user is a patient, the patient's doctor, or a health professional.

20. The system of claim 14, wherein the predictive biomarker changes are modeled using a three-dimensional model.
